(19) United States
(12) Patent Application Publication
(10) Pub. No.: US 2026/0284422 A1
(43) Pub. Date: Sep. 24, 2026

(54) BRAIN TUMOR TREATMENT OR PREVENTION OF RECURRENCE WITH LOW INTENSITY AND VARIABLE FREQUENCY MAGNETIC STIMULATION

(71) Applicant: ACTIPULSE NEUROSCIENCE INC., Cambridge, MA (US)

(72) Inventors: Gabriel Villafuerte, Cambridge, MA (US); Adrien Châtillon, Cambridge, MA (US); Irina Vazquez Villaseñor, Cambridge, MA (US)

(21) Appl. No.: 19/478,790

(22) PCT Filed: Apr. 25, 2024
(86) PCT No.: PCT/US2024/026145

§ 371 Date: Oct. 24, 2025

Related U.S. Application Data
(60) Provisional application No. 63/461,987, filed on Apr. 26, 2023.

(51) Int. Cl. A61N 2/00; A61N 2/02

(52) U.S. Cl. CPC A61N 2/006; A61N 2/002; A61N 2/02

(57) ABSTRACT

The present disclosure provides, in part, methods of treatment and prevention of recurrence for brain tumors, e.g., without limitation, GBM comprising applying repetitive transcranial magnetic stimulation (rTMS) therapy to a patient in need thereof. The present disclosure also provides devices that generate a low intensity magnetic field and variable frequencies.

### CROSS-REFERENCE TO RELATED APPLICATIONS

This application claims the benefit of and priority to U.S. Provisional Application No. 63/461,987, filed Apr. 26, 2023, the entirety of which is hereby incorporated by reference.

### FIELD

The present disclosure provides, in part, clinical applications of a non-invasive brain stimulation, e.g., repetitive Transcranial Magnetic Stimulation (rTMS), as it pertains to neuromodulation in the treatment and/or prevention of recurrence of brain tumors.

### BACKGROUND

GBM is the most common and aggressive type of primary brain tumor due to its fast-growing nature, its ability to affect surrounding brain regions and its resistance to treatment (van Solinge et al. 2022; Weller et al. 2021). GBM comprises up to 50% of all gliomas and in most cases, its cause is unknown. GBM derives from neuroglial stem or progenitor cells that have the ability to infiltrate adjacent normal brain tissue; moreover, a specific stem-like cell population known as glioma stem cells (GSCs) are present in GBM and confer this type of tumor the distinctive resistance to chemotherapy and radiotherapy due to the GSCs ability of self-renewal and tumorigenicity (Rong et al. 2022).

The 2021 World Health Organization (WHO) Classification of Tumors of the Central Nervous System considered not only histopathological features but also the most recently described molecular characteristics to update the GBM classification criteria (Louis et al. 2021; Torp et al. 2022; Weller et al. 2021). Based on the 2021 report, tumors of astrocytic origin are now grouped depending on the presence of isocitrate dehydrogenase (IDH) mutations and are graded II, III, or IV: those without IDH mutations are called glioblastomas IDH-wildtype (grade IV), while those with IDH mutations are termed astrocytomas IDH-mutants (grades II, III, IV). These two categories are included in the adult-type diffuse gliomas classification, together with oligodendrogliomas (IDH-mutant, 1p/19q-codeleted). Glioblastoma IDH-wildtype Grade IV is characterized by necrosis and/or microvascular proliferation, however in cases where these histological features are lacking, there are other criteria that can help to determine whether an astrocytic diffuse glioma should be classified as glioblastoma IDH-wildtype Grade IV. These criteria rely on the molecular changes seen in the tumor and comprise the concurrent gain of whole chromosome 7 and loss of whole chromosome 10 (+7/−10), mutations in the Telomerase Reverse Transcriptase (TERT) promoter and epidermal growth factor receptor (EGFR) amplification. The presence of any of the latter molecular changes, or a combination of these, supports the upgrading of the diagnosis of lower-grade IDH-wildtype astrocytomas to glioblastoma IDH-wildtype Grade IV (Melhem et al. 2022). In summary, WHO 2021 classification clearly separates adult from pediatric-type gliomas and identifies as GBM the most aggressive form of IDH-wildtype diffuse adult-type astrocytoma.

GBM usually localizes to the supratentorial compartment, and is most frequently present in the frontal lobe, but can also locate to the temporal, parietal and occipital lobes; GBM can also originate in the brainstem or cerebellum, although these cases are rare (Grochans et al. 2022). GBM differentiates from lower-grade tumors by necrosis and/or hypoxia induced microvascular hyperplasia. Research on the genetic and molecular changes involved in the development of GBM have identified mutations in different genes, including IDH, EGFR, TERT, ATRX, TP53, B-RAF (V600E mutation), GATA4, FGFR1, WT1, and PTEN, that provide information on tumor type, prognosis, and tumor progression (Le Rhun et al. 2019). These mutations also play an important role in the pathogenesis of GBM. For instance, IDH mutations cause loss and gain of function of IDH enzymatic activity, which is an essential component of the citric acid cycle. As a result, mutations in the IDH gene promote aberrant accumulation of the oncometabolite 2-hydroxyglutarate (HG) and carcinogenesis (Melhem et al. 2022; Alzial et al. 2022). The EGFR pathway, also affected in GBM through EGFR amplification, is involved in mechanisms that promote cell proliferation, migration and adhesion; similarly, TERT mutations cause an increase in TERT promoter expression, resulting in increased telomerase activity and telomere elongation, key mechanisms needed for tumor formation (Grochans et al. 2022).

Genetic changes are not the only processes involved in the development and progression of GBM. Other molecular changes that have been studied in GBM and other types of tumors are related to their mechanical properties, specifically tumor stiffness, and the role that these properties play in tumor cell proliferation (Ishihara & Haga 2022). One of the mechanisms that have been investigated is linked to PIEZO1, a transmembrane protein that functions as a mechanosensitive cation channel, inducing cationic non-selective activated currents in response to mechanical stimuli. In the context of breast cancer, PIEZO1 has been shown to regulate tumor development by increasing motility in response to tumor cell growth (Li et al. 2015). Together with its homologue PIEZO2, PIEZO1 channels are activated by these mechanical signals to then interact with multiprotein structures that regulate the cytoskeleton and facilitate tumor development. PIEZO1 gene expression in GBM (grade IV) is significantly higher compared to low grade gliomas (grade II and III); at the protein level, GBM also had the highest expression of PIEZO1, indicating that its participation in the mechanic properties of GBM that are linked to its tumorigenicity is relevant and should be considered when designing alternate therapeutic strategies (Chen et al. 2018; Zhou et al. 2020).

GBM accounts for 54% of all malignant adult brain and other CNS tumors that are diagnosed in the United States. It is 1.59 times more common in males and its incidence rate increases exponentially beyond 40 years of age, with the highest rates occurring among those aged 75 to 84 years. Between 2008 and 2017, incidence rates remained stable; however, the median overall survival for patients diagnosed with GBM continues to be low these days, ranging from 14.6 to 20.5 months, with the prognosis being worse for elderly patients (Miller et al. 2021).

The incidence rate in non-Hispanic white adults (5.1 cases per 100,000) is higher to the ones seen in non-Hispanic black adults (2.5 cases per 100,000), American Indian/Alaska Native adults (2.6 cases per 100,000) and in Asian/Pacific Islander adults (1.6 cases per 100,000). In terms of survival, it is higher for females that have received the standard care of treatment; survival rates based on race or ethnicity are variable most likely due to variations in health care access and case severity, however, a study performed in 2018 looking at glioma incidence in adults in the United States found a poorer outcome after diagnosis and lower survival rate for non-Hispanic White adults compared to other groups (Miller et al. 2021).

The only validated risk factor for GBM is exposure to ionizing radiation in medium to high doses. The majority of the GBM cases are sporadic, with only 1-2% of adult glioma linked to a Mendelian inherited syndrome (Grochans et al. 2022; Miller et al. 2021).

GBM has no cure. The current standard of care for GBM includes maximal surgical resection, radiotherapy and alkylating chemotherapy, but despite advances in treatment, the prognosis of patients remains poor as a result of GBM high recurrence after standard multimodality treatment (Le Rhun et al. 2019; Melhem et al. 2022). The presence of GSCs confers GBM resistance to chemotherapy and radiotherapy, and its tendency to invade adjacent normal brain tissue prevents complete resection of the tumor (Rong et al. 2022). The use of molecular profiling has allowed for a better understanding of GBM but there are still several limitations that have to be addressed in the development of effective therapies; these include the intra- and inter-tumor molecular heterogeneity and the availability of brain-targeted therapies that can cross the blood brain barrier (BBB). Newer approaches to treatment that are currently under study include cytoreductive surgery, laser interstitial thermal therapy (LITT), the use of fluorescence detection techniques and intraoperative MRI (iMRI) to maximize resection, MRI-guided Radiation Therapy, microtubule-targeting agents (MTAs), and focused ultrasound (FUS) for BBB disruption to facilitate targeted drug delivery (Melhem et al. 2022).

NiBS techniques are based on the application of transcranial electrical or magnetic fields to alter neuronal activity. This group of techniques has been widely used in clinical and research settings due to its efficacy and safety in the treatment of a variety of psychiatric and neurological conditions; based on this, NiBS could be used to treat brain cancers, such as GBM, but this potential has not been yet explored. Transcranial magnetic stimulation (TMS) is a NiBS technique with a high safety profile that has been used as a neurologic and psychiatric treatment; it is also employed as a brain imaging technique (navigated TMS) for GBM pre- and post-operative functional mapping (Motomura et al. 2020), but there is no experimental data of its direct effects on GBM. On the other hand, evidence of the effects of transcranial direct current stimulation (tDCS) on GBM shows that tDCS transiently reduces tumor perfusion and could impact tumor growth (Sprugnoli et al. 2019). It is important to note that the use of repetitive TMS as a treatment for neurocognitive diseases and disorders is expensive, requires a clinical setting, devices are hard to transport and complex to use, they require external coolers, and their use may have severe adverse effects such as seizures. These disadvantages are the consequence of current TMS technology, which uses frequencies from 1 to 50 Hz and intense magnetic fields of around 10 million milligauss (i.e., around 1 Tesla), which is makes them inaccessible to patients as well as safe and easy to use from home.

Tumor-treating fields (TTF) is a non-invasive technology that has been investigated for the treatment of cancer over the past 10 years. TTF creates low-intensity (1-3 V/cm) alternating electric fields and intermediate frequency (100-300 KHz) to disrupt cell division and limit proliferation of cancer cells. This technique was approved by the FDA as a therapeutic option for recurrent and refractory GBM in 2011, and for newly diagnosed GBM in 2015 (Rominiyi et al. 2021). To date, there are commercially available, wearable, portable TTF devices, such as OPTUNE, for the treatment of GBM; however, these devices still have limitations that must be addressed. For instance, TTF side effects include headache and presence of irritant or allergic contact dermatitis on the scalp as a consequence of prolonged exposure to the hydrogel and adhesive. The cost is also something to take into consideration, since the rent of a TTF device could be around 21,000 USD per month.

There is a need for improved treatments for GBM that are of non-invasive nature that prevent tumor recurrence and have a positive impact on patients' survival and quality of life. There is also a need for devices and methods that employ a non-invasive brain stimulation, e.g., rTMS, without using large magnetic fields, and without focusing the magnetic waves at a certain point in the brain. There is a further need for a user-friendly device that is accessible, portable, appropriate for home use, and is safe to use without the need of a medical professional to apply it.

### SUMMARY

Accordingly, the present disclosure provides low intensity and variable frequency non-invasive brain stimulation, e.g., rTMS, methods to safely and effectively treat or prevent recurrence of a brain tumor, e.g., without limitation, GBM in a convenient manner, e.g., directly from home and without clinical supervision.

In aspects, the present disclosure provides a non-invasive brain stimulation, e.g. rTMS, method for treating or preventing recurrence of a brain tumor, e.g. without limitation, GBM comprising repetitively applying a magnetic pulse to the scalp (e.g., head surface) of a patient in need thereof thereby stimulating neurons in the brain of the patient, wherein the magnetic pulse is applied (a) repetitively over the patient's brain (e.g., without limitation, the patient's left prefrontal dorsolateral cortex); and (b) at a frequency of about 10 Hz to about 500,000 Hz and an intensity of about 5,000 milligauss to about 1,000,000 milligauss (about 0.0005 Tesla to about 0.1 Tesla).

In embodiments, the brain tumor is an astrocytoma. In embodiments, the brain tumor is a glioma. In embodiments, the brain tumor is a pilocytic astrocytoma (grade 1), diffuse astrocytoma (grade 2), anaplastic astrocytoma (grade 3), or glioblastoma (grade 4). In embodiments, the brain tumor is a glioblastoma. In embodiments, the glioblastoma is a glioblastoma IDH-wildtype (grade IV). In embodiments, the glioblastoma is characterized by an IDH mutation, e.g., an astrocytoma IDH-mutants (grades II, III, IV). In embodiments, the glioblastoma is an adult-type diffuse glioma. In embodiments, the glioblastoma is an oligodendroglioma (IDH-mutant, 1p/19q-codeleted). In embodiments, the glioblastoma is glioblastoma multiforme (GBM). In embodiments, the glioblastoma is a primary GBM. In embodiments, the glioblastoma is a secondary GBM. In embodiments, the GBM localizes to the supratentorial compartment. In embodiments, the GBM localizes to and/or originates in the frontal lobe. In embodiments, the GBM localizes to and/or originates in the temporal, parietal and/or occipital lobes. In embodiments, the GBM localizes to and/or originates in the brainstem or cerebellum. In embodiments the brain tumor is characterized by mutations in one or more of the following genes: IDH, EGFR, TERT, ATRX, TP53, B-RAF (V600E mutation), GATA4, FGFR1, WT1, and PTEN. In embodiments the brain tumor is characterized by higher gene expression and/or activity of PIEZO1, e.g., as compared to a non-cancerous state and/or low grade gliomas (e.g. grade II and III). In embodiments the brain tumor patient has been exposed to ionizing radiation in medium to high doses. In embodiments the present treatment methods are used in combination with one or more of surgical resection, radiotherapy and alkylating chemotherapy.

In embodiments, the patient is afflicted with one or more symptoms selected from intracranial hypertension, headaches, seizures, sensory disturbances, neurocognitive symptoms, and motor symptoms. In embodiments, the symptoms originate from cancer, e.g., from being afflicted with GBM.

In embodiments, the present disclosure contemplates applying a magnetic pulse using rTMS to a patient afflicted with a brain tumor, e.g., without limitation, GBM. In embodiments, the present intervention provides applying rTMS to a patient with a brain tumor, e.g. without limitation, GBM, having one or more of the following adult-type diffuse gliomas subtypes, as determined by the 2021 WHO Classification of Tumors of the Central Nervous System histopathological and molecular grading and The Consortium to Inform Molecular and Practical Approaches to CNS Tumor Taxonomy-Not Official WHO (cIMPACT-NOW): glioblastoma, IDH-wildtype, grade IV, including those classified as diffuse astrocytic glioma, IDH-wildtype, with molecular features of glioblastoma, grade IV, which have been upgraded from lower-grade (II and III) IDH-wildtype astrocytomas to glioblastoma IDH-wildtype Grade IV based on (+7/−10), TERT promoter mutation and EGFR amplification.

In embodiments, the present disclosure contemplates applying a magnetic pulse using rTMS to a patient afflicted with a brain tumor, e.g., without limitation, GBM. In embodiments, the patient with a brain tumor, e.g., without limitation, GBM, has one or more of the following symptoms: the individual presents with intracranial hypertension, headaches, seizures, sensory disturbances, neurocognitive symptoms, motor symptoms.

In embodiments treating the brain tumor, e.g., without limitation, GBM, increases overall survival (OS) in GBM patients maintaining an acceptable safety profile. OS is defined as the time elapsed from randomization in a clinical trial until death from any cause.

In embodiments treating the brain tumor, e.g., without limitation, GBM, improves endpoints based on tumor assessments, including disease free-survival (DFS), event-free survival (EFS), objective response rate (ORR), complete response (CR), time to progression (TTP), progression-free survival (PFS) and time to treatment failure (TTF). DFS is defined as the time elapsed between randomization and brain tumor, e.g. without limitation, GBM, recurrence or death from any cause while EFS is the time elapsed from randomization to any of the following events: progression of brain tumor, e.g. without limitation, GBM, that precludes surgery (tumor resection), local or distant recurrence, or death from any cause. ORR is the proportion of patients with tumor size reduction of a predefined amount and for a minimum time period; this is measured from the time of initial response until documented tumor progression. CR is defined as no detectable evidence of tumor measured through imaging studies or histopathological assessment. TTP is the time from randomization until objective tumor progression and does not include deaths, while PFS is the time from randomization until objective tumor progression or death. TTF is a combined endpoint measuring time from randomization to discontinuation of treatment for any reason (disease progression, treatment toxicity, and death).

In embodiments treating the brain tumor, e.g., without limitation, GBM, prevents recurrence treatment with an additional therapeutic agent and/or additional therapeutic modality.

In embodiments treating the brain tumor, e.g., without limitation, GBM, prevents recurrence after surgical resection as measured by the efficacy endpoints PFS and OS.

In embodiments treating the brain tumor, e.g., without limitation, GBM, slows tumor progression as defined by the Response Assessment in Neuro-Oncology (RANO) criteria for high-grade and lower-grade gliomas to assess the response to a treatment, as well as progression.

In embodiments treating the brain tumor, e.g., without limitation, GBM, involves symptomatic improvement in patients. This improvement can be measured by specific endpoints such as symptom improvement/palliation and time to progression of cancer symptoms.

In embodiments treating the brain tumor, e.g. without limitation, GBM, prevents or diminishes side effects of current chemotherapy treatment with temozolomide, such as nausea, vomiting, weakness, unusual bleeding, bruising, blistering, peeling, red skin rash, low levels of blood cells, hair loss, and loss of appetite. In embodiments treating the brain tumor, e.g. without limitation, GBM, prevents or diminishes side effects of radiation therapy, such as fatigue, radiation dermatitis, hair loss, headaches, nausea, vomiting, hair loss, hearing loss, seizures, endocrine problems, and problems with memory and speech.

In embodiments, treating the brain tumor, e.g., without limitation, GBM, increases health-related quality of life (HRQOL) of patients as assessed by patient-reported outcome (PROs) measures that encompass social, financial, psychosocial and physical activities. The specific tools that can be used for evaluating HRQOL encompass the EORTC questionnaires QLQ-C30 (Quality of life of cancer patients), QLQ-BN20 (Quality of life of brain cancer patients), and QLQ-FA12 (Cancer related fatigue).

Initial diagnostic workup in patients with the brain tumor, e.g. without limitation, GBM, includes neurological and neurocognitive assessment using one or more of the following scales: The Neurological Assessment in Neuro-Oncology (NANO), Mini Mental State Examination (MMSE), Montreal Cognitive Assessment (MoCA), Karnofsky Performance Score (KPS), Cambridge Neuropsychological Test Automated Battery (CANTAB), Oxford Cognitive Screen (OCS)-Bridge assessment, National Institutes of Health (NIH) Toolbox. In relation to brain imaging techniques: Magnetic Resonance Imaging (MRI), including T2-weighted, T2-weighted fluid-attenuated inversion recovery (FLAIR) sequences and 3D T1-weighted sequences, Positron Emission Tomography (PET), electroencephalography (EEG). Detection of molecular markers from biopsies: IDH mutations, 1p/19q codeletion, MGMT promoter methylation, mutations in the H3F3A, platelet-derived growth factor receptor-α (PDGFRA), TERT promoter mutation. In embodiments, treating or preventing recurrence of the brain tumor, e.g., without limitation, GBM, modifies one of the mentioned assessments.

Patients with diffuse glioma may develop alterations in different cognitive domains, associated with the affected area and the morphology of the tumor. Also, it has been found that diffuse gliomas can involve larger brain networks. In addition, chemotherapy and radiotherapy used for treatment may contribute to the deterioration of cognitive functions. Together, all these factors can affect cognitive functions that condition the quality of life of those who suffer from it.

In embodiments treating the brain tumor, e.g., without limitation, GBM, improves endpoints based on tumor assessments, including disease free-survival (DFS), event-free survival (EFS), objective response rate (ORR), complete response (CR), time to progression (TTP), progression-free survival (PFS) and time to treatment failure (TTF). DFS is defined as the time elapsed between randomization and brain tumor, e.g. without limitation, GBM, recurrence or death from any cause while EFS is the time elapsed from randomization to any of the following events: progression of the brain tumor, e.g. without limitation, GBM, that precludes surgery (tumor resection), local or distant recurrence, or death from any cause. ORR is the proportion of patients with tumor size reduction of a predefined amount and for a minimum time period; this is measured from the time of initial response until documented tumor progression. CR is defined as no detectable evidence of tumor measured through imaging studies or histopathological assessment. TTP is the time from randomization until objective tumor progression and does not include deaths, while PFS is the time from randomization until objective tumor progression or death. TTF is a combined endpoint measuring time from randomization to discontinuation of treatment for any reason (disease progression, treatment toxicity, and death).

In embodiments treating the brain tumor, e.g., without limitation, GBM, prevents recurrence after surgical resection as measured by the efficacy endpoints PFS and OS.

In embodiments treating the brain tumor, e.g., without limitation, GBM, slows tumor progression as defined by the Response Assessment in Neuro-Oncology (RANO) criteria for high-grade and lower-grade gliomas to assess the response to a treatment, as well as progression.

In embodiments, methods of rTMS herein reduce, impair, and/or decreases tumor growth, migration, invasiveness, and/or metastasis phenotypes throughout the patient's brain without substantially adversely affecting healthy tissues.

### DESCRIPTION OF THE DRAWINGS

FIG. 1 depicts a helmet with an array of coils of a non-limiting example of the rTMS device

FIG. 2 depicts a stimulation device having an anesthesia platform with anesthesia masks and tubing for up to five mice. The stimulation coils are attached to the platform and connected to the HFLI TMS device. Mice under general anesthesia are carefully placed face up, with their heads on top of the coils, and are secured to prevent movement.

FIG. 3 depicts a pictorial representation of illustrative methods for investigating the effect of rTMS on BBB permeability of the brain using Evans blue dye (EBD) in vivo with representative images of resected brains used for ex vivo quantification.

FIG. 4 depicts a pictorial representation of the internal brain staining of representative brain sectioning obtained from EBD-injected mice without rTMS intervention (left) and with rTMS intervention (right). The darker, brown regions within the tissue, as well as the outlining of blood-brain barrier (BBB) vessels, is more prominent in the sectioning obtained from mice that underwent rTMS. Scale bar is about 2.5 mm.

FIG. 5 depicts a graphical representation of the quantification of brain sectioning area with Evans blue staining in mice without rTMS intervention (left) and those with rTMS intervention (right). rTMS intervention caused a statistically significant increase in staining by Evans blue dye signifying a significant increase in BBB permeability.

FIG. 6 depicts a pictorial representation of illustrative methods for investigating the effect of rTMS on BBB permeability of the brain using a fluorescent CXCR1 mouse model with representative in vivo fluorescence confocal microscopy imaging demonstrating colocalization of dextran and glial cells after rTMS exposure. Scale bars are 50 μm.

FIG. 7 depicts a graphical representation of the quantification of the in vivo fluorescence imaging demonstrating the effect of rTMS on BBB permeability.

FIG. 8 depicts a pictorial representation of illustrative methods for investigating the effects of rTMS on glioblastoma cells in vitro. For reference, at point 10-2, on the x-axis the top curve is “no stimulation” (sham treatment) and the bottom curve is 1 hr. rTMS treatment.

FIG. 9 depicts representative fluorescence confocal microscopy imaging and a graphical representation quantifying the results of rTMS on the effect of the migration of glioblastoma cells, e.g., to form spheroids in vitro. rTMS was shown to lead to a statistically significant decrease in 3D spheroid formation in 0.6% agarose (consistent with the stiffness of brain tissue) (p<0.013). NT=sham treatment.

FIG. 10 depicts representative confocal microscopy imaging and a graphical representation quantifying the results of rTMS on the effect of the invasion and wound healing of glioblastoma cells in vitro. rTMS was shown to lead to a statistically significant decrease in invasion and wound healing of glioblastoma cells at 24 hrs. (p=0.01). NT=sham treatment.

### DETAILED DESCRIPTION

In aspects, the present disclosure provides, in part, methods for the treatment of a brain tumor, e.g. without limitation, GBM, or the prevention of recurrence of a brain tumor, e.g. without limitation, GBM, including but not limited to reduction of intracranial hypertension, headaches, seizures, sensory disturbances, neurocognitive symptoms and motor symptoms. In embodiments, treating a brain tumor, e.g., without limitation, GBM, or preventing a brain tumor, e.g., without limitation, GBM, recurrence comprises reduction in tumor size, increase in overall survival, benefits in quality of life and tumor related assessments.

In aspects, the present disclosure provides a method using high frequency, low intensity transcranial magnetic stimulation and/or non-invasive brain stimulation using variable frequency and low intensity magnetic fields for the treatment or prevention of recurrence of a brain tumor, e.g., without limitation, GBM.

Repetitive Transcranial Magnetic Stimulation (rTMS)

In embodiments, the present disclosure provides methods for using non-invasive brain stimulation.

In embodiments, the present disclosure provides for using transcranial magnetic stimulation (TMS) in a non-invasive and non-painful method to stimulate the cerebral cortex. In embodiments, the present disclosure contemplates a device that generates low-intensity magnetic fields and high frequencies. In embodiments, the magnetic field could be created by an alternate current or a pulsed current. In embodiments, rTMS activates or inhibits cortical activation by generating a magnetic field that, by Faraday's principle, generates an electrical field inside the tumor. In embodiments, the magnetic field is produced when an electric current passes through a coil. Further, in embodiments, in order to generate an electric field inside the tumor, the magnetic field starts and finishes rapidly. Without wishing to be bound by theory, if the magnetic field intensity stays constant too long, no electrical changes occur inside the tumor. In addition, it is contemplated that when making use of magnetic fields of lower magnitude, neither a voltage transformer nor high currents are necessary. Indeed, the present disclosure contemplates a device that decreases potential adverse effects, facilitates its transport, and does not require external coolers for the operation.

In embodiments, the disclosure generates an alternate current and passes it through a copper coil. In embodiments, the magnetic field is not strong enough to generate an electric current inside the brain. In embodiments, the disclosure achieves its anti tumoral effect by a mechanism of action different from electromagnetic induction.

In embodiments, the present disclosure produces a magnetic field that not only activates or inhibits cells inside the brain of patients with a brain tumor, e.g., without limitation, GBM, but also synchronizes cortical neural firing to the frequency of the magnetic pulses. This entrainment effect will change underlying oscillatory activity of neurons and, in consequence, modify the possible trophic relationship between tumor and brain activity.

In embodiments, the present disclosure provides for using transcranial magnetic stimulation (TMS), wherein the magnetic pulse is applied at a frequency of about 10 to about 500,000 Hz, about 10 to about 400,000 Hz, about 10 to about 200,000 Hz, about 10 to about 100,000 Hz, about 10 to about 50,000 Hz, about 10 to about 25,000 Hz, about 10 to about 12,500 Hz, about 10 to about 6,000 Hz, about 10 to about 3000 Hz, about 10 to about 2000 Hz, about 10 to about 1000 Hz, about 10 to about 500 Hz, about 10 to about 200 Hz, about 10 to about 100 Hz, or about 10 to about 20 Hz.

In embodiments, the present disclosure provides for using transcranial magnetic stimulation (TMS), wherein the magnetic pulse is applied at a frequency of about 200 Hz.

In embodiments, the present disclosure provides for using transcranial magnetic stimulation (TMS), wherein the magnetic field is applied at an intensity of about 0.0005 Tesla to about 0.1 Tesla, or about 0.005 to about 0.01 Tesla, or about 0.0005 to about 0.001 Tesla.

In embodiments, the present disclosure provides for using transcranial magnetic stimulation (TMS), wherein the magnetic field is applied at an intensity of about 0.01 Tesla.

In embodiments, the present disclosure provides for using transcranial magnetic stimulation (TMS), wherein the magnetic pulse is applied at a frequency of about 200 Hz and at an intensity of about 0.01 Tesla.

In embodiments, the present disclosure provides for using transcranial magnetic stimulation (TMS) at about 1,000 to about 200,000 milligauss, or about 5,000 to about 150,000 milligauss, or about 7,000 to about 120,000 milligauss. In embodiments, the present disclosure provides for using transcranial magnetic stimulation (TMS), wherein the magnetic pulse is applied at an intensity of about 1,000 to about 200,000 milligauss, or about 1,000 to about 150,000 milligauss, or about 1,000 to about 100,000 milligauss, or about 1,000 to about 50,000 milligauss, or about 50,000 to about 150,000 milligauss, or about 10,000 to about 150,000 milligauss. In embodiments, the magnetic pulse is applied at an intensity of about 1,000 milligauss, about 2,000 milligauss, about 3,000 milligauss, about 4,000 milligauss, about 5,000 milligauss, about 6,000 milligauss, about 7,000 milligauss, about 8,000 milligauss, about 9,000 milligauss, about 10,000 milligauss, about 11,000 milligauss, about 12,000 milligauss, about 13,000 milligauss, about 14,000 milligauss, about 15,000 milligauss, about 16,000 milligauss, about 17,000 milligauss, about 18,000 milligauss, about 19,000 milligauss, about 20,000 milligauss, about 50,000 milligauss, about 100,000 milligauss, about 150,000 milligauss or 200,000 milligauss.

In embodiments, the present disclosure provides for using transcranial magnetic stimulation (TMS), wherein the magnetic pulse is applied at an intensity of about 0.0001 to about 0.002 Tesla, or about 0.0005 to about 0.0015 Tesla, or about 0.0007 to about 0.0012 Tesla. In embodiments, the magnetic pulse is applied at an intensity of about 0.0001 Tesla, about 0.0002 Tesla, about 0.0003 Tesla, about 0.0004 Tesla, about 0.0005 Tesla, about 0.0006 Tesla, about 0.0007 Tesla, about 0.0008 Tesla, about 0.0009 Tesla, about 0.001 Tesla, about 0.0011 Tesla, about 0.0012 Tesla, about 0.0013 Tesla, about 0.0014 Tesla, about 0.0015 Tesla, about 0.0016 Tesla, about 0.0017 Tesla, about 0.0018 Tesla, about 0.0019 Tesla, or about 0.002 Tesla.

In embodiments, the present disclosure provides for using transcranial magnetic stimulation (TMS), wherein the magnetic pulse generates an electric field of about 0.1 to about 10 V/m², about 0.5 to about 5 V/m², or about 0.5 to about 1.5 V/m². In embodiments, the magnetic pulse generates an electric field of about 1 V/m², about 2 V/m², about 3 V/m², about 4 V/m², about 5 V/m², about 6 V/m², about 7 V/m², about 8 V/m², about 9 V/m², about 10 V/m², about 20 V/m² or about 30 V/m²

In embodiments, the rTMS treatment method is undertaken once, or twice, or thrice, or four times daily. In embodiments, the method is undertaken once, twice, or thrice daily. In embodiments, the rTMS treatment method is undertaken for greater than about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In embodiments, the method is undertaken for about 15 to about 60 minutes. In embodiments, the method is undertaken for about 30 minutes. In embodiments, the rTMS treatment method comprises magnetic pulses that are applied about 200 to about 1000 times per second, or about 200 to about 900 times per second, or about 200 to about 800 times per second, or about 200 to about 700 times per second, or about 200 to about 600 times per second, or about 200 to about 500 times per second, or about 200 to about 400 times per second, or about 200 to about 300 times per second, or about 300 to about 1000 times per second, or about 300 to about 900 times per second, or about 300 to about 800 times per second, or about 300 to about 700 times per second, or about 300 to about 600 times per second, or about 300 to about 500 times per second, or about 300 to about 400 times per second. In embodiments, the rTMS treatment method comprises magnetic pulses that are applied about 300 to about 400 times per second. In embodiments, the pulses are applied about 10 times per second, about 20 times per second, about 30 times per second, applied about 40 times per second, about 50 times per second, about 80 times per second, about 300 times per second, about 310 times per second, about 320 times per second, about 330 times per second, about 340 times per second, about 350 times per second, about 360 times per second, about 370 times per second, about 380 times per second, about 390 times per second, or about 400 times per second. In embodiments, the pulses last for about two seconds, about three seconds, about four seconds, or about five seconds. In embodiments, the pulses last for about four seconds, followed by one second without pulsing. In embodiments, a pulse is applied for a period of four seconds, followed by a one second pause in which no pulse is applied. In embodiments, the pulses are applied continuously during the whole time of the session without interruption.

In embodiments, the method referred to is applied continuously during the day without interruption. In embodiments, the method is applied for about 6 hours per day, about 12 hours, about 18 hours, or about 24 hours per day.

In embodiments, the rTMS treatment method contemplated by the present disclosure is applied chronically. For example, in embodiments, the treatment is applied for greater than about 1 month, about 2 months, about 3 months, about 4 months, about 5 months, about 6 months, about 7 months, about 8 months, about 9 months, about 10 months, about 11 months, or about 1 year, or about 2 years, or about 3 years, or about 5 years, or about 10 years. In embodiments, the treatment is applied for the life of the patient. In embodiments, the treatment is self-applied.

In embodiments, the rTMS treatment method contemplated by the present disclosure is applied to the patient until symptoms, such as those described herein, improve or diminish. For example, the patient may undergo the treatment and experience a decrease in tumor size and hope to terminate the treatment. The patient may re-start the treatment if tumor size increases again. Alternatively, the patient may undergo the treatment and experience a slowing or halting of tumor growth, but not an improvement. In such a situation, in embodiments, the patient continues treatment chronically to mitigate or prevent further growth of a brain tumor, e.g., without limitation, GBM.

rTMS Device

In embodiments, the present disclosure provides devices and equipment for TMS, comprising an electromagnetic field generator and a coil that emits said electromagnetic field. In embodiments, the magnetic field is produced when an electric current passes through a coil. In embodiments, pulses of TMS are applied repeatedly with a frequency and a determined magnitude over a cortical area in order to modulate the cerebral cortex activity in the long term. In embodiments, pulses of TMS are applied repeatedly at physiologically measured frequencies over a cortical area in order to synchronize the firing of neurons in brain; in this embodiment, entraining neurons affect the trophic effect of neurons over tumor cells. In embodiments, magnetic pulses are applied at higher frequencies (above 500 Hz) to inhibit growth and/or invasiveness in the peripheral area of the tumor. In embodiments, magnetic pulses are applied at several frequencies to affect the growth and/or invasiveness of tumoral cells inside the brain.

It will be appreciated that the methods of the present disclosure are applied via a device capable of administering the transcranial magnetic stimulation. Indeed, in embodiments, the TMS is applied using a device that is suitable for conducting electric current through a coil, thus generating a magnetic field. In embodiments, the device is suitable for home use and is optionally portable. In embodiments, the device comprises a touch screen and optionally includes “digital health” applications and tele-health solutions. In embodiments, the device is connected via Bluetooth to a smartphone.

In a non-limiting example, the present disclosure contemplates a device that generates a low intensity pulsed magnetic field and variable frequencies. In embodiments, the device is a transcranial magnetic stimulation system able to generate pulsed magnetic fields in a frequency range from 10 Hz to 500,000 Hz. In embodiments, the device is a transcranial magnetic stimulation system that generates a magnetic field with an intensity from 5,000 milligauss to 1,000,000 milligauss.

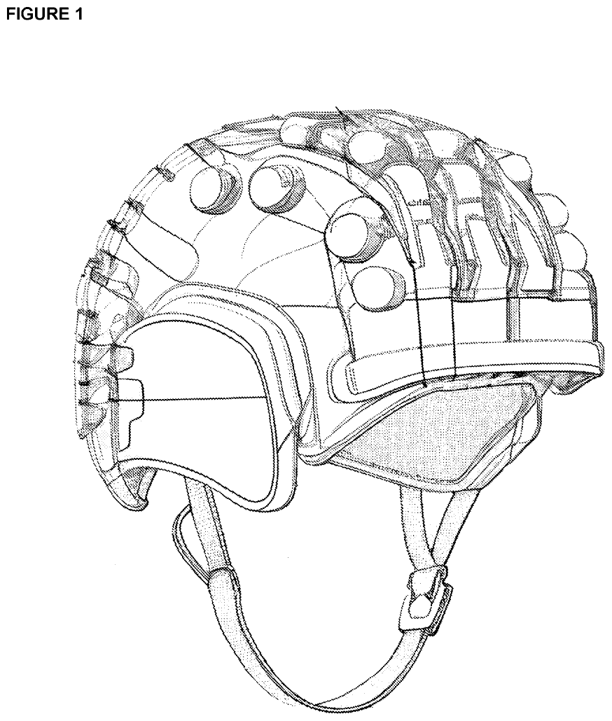

In embodiments, the TMS-generating device comprises three parts: a portable battery pack, an array of coils located inside a helmet and an electronic unit. In embodiments, the electronic unit is a PCB (print cardboard) in which all the electronic components responsible to provide the electromagnetic waves to the coil by the connector are soldered. In embodiments, the TMS-generating device is a wearable device, e.g., as depicted in FIG. 1.

In embodiments, the electronic circuit is composed of four parts: (1) a pulse generator suitable for pulsing an electric current and/or magnetic field from 10 Hz to 500,000 Hz, (2) an amplifier of electrical current (3) control and monitoring of critical variables (4) and a battery charging circuit. The electric current passes 10 to 500,000 times per second through the coil and the flux is not interrupted during the duration of the stimulation session. In embodiments, the circuit is able to generate a pulsed magnetic field with an approximate intensity of 5,000 milligauss to 1,000,000 milligauss. In embodiments, the board has transistors, resistors, heat sinks, electrolytic capacitors, and diodes in order to generate the pulse to the given frequency.

In embodiments, an array of coils that transmits the magnetic pulse is located in a wearable device, e.g., a helmet, that covers the skull. In embodiments, the device comprises multiple coils of 3-6 cm in diameter built from 50-100 turns of copper wire 99.5% pure, gauge 24-26 AWG. In embodiments, a double coverage of insulating material covers the coil winding.

In embodiments, the array of coils that comprises the device is a helmet or headband that allows to place the coils in the correct position over the skull. In embodiments, this helmet or headband can be fabricated from plastic, steel, aluminum or other materials. In embodiments, the headband can have fabric in the side that fits the skull in order to comfortably fit the subject's head.

In embodiments, a battery or set of batteries are used as a power source. The power source supplies the electronics and coil with the necessary current to operate the device and generate the magnetic pulses intended to be applied over the skull. In embodiments, the battery or set of batteries can be easily changed in order to allow the user to use the disclosure for a longer period of time.

### Methods of Treatment and Use

In aspects, the present disclosure contemplates methods for treating a brain tumor, e.g., without limitation, GBM, by halting its progression using non-invasive brain stimulation, e.g., rTMS. In embodiments, the method of rTMS comprises repetitively applying a magnetic pulse to the scalp of a patient in need thereof, thereby affecting tumoral cells in the brain of the patient, wherein the magnetic pulse is applied (a) repetitively over the patient's brain; and (b) at a frequency of about 10 Hz to about 500,000 Hz and an intensity of about 5,000 milligauss to about 1,000,000 milligauss. In embodiments, methods of the present disclosure are suitable for home use and can be performed without the need for a medical professional.

In aspects, the present disclosure contemplates methods for preventing after treatment with an additional therapeutic agent and/or additional therapeutic modality.

In aspects, the present disclosure contemplates methods for preventing a brain tumor, e.g., without limitation, GBM, recurrence after surgical resection. In aspects, the present disclosure contemplates methods for preventing a brain tumor, e.g., without limitation, GBM, recurrence after surgical resection using non-invasive brain stimulation, e.g., rTMS. In embodiments, the method of rTMS comprises repetitively applying a magnetic pulse to the scalp of a patient in need thereof, thereby affecting tumoral cells in the brain of the patient, wherein the magnetic pulse is applied (a) repetitively over the patient's brain and (b) at a frequency of about 10 Hz to about 500,000 Hz and an intensity of about 5,000 milligauss to about 1,000,000 milligauss. In embodiments, methods of the present disclosure are suitable for home use and can be performed without the need for a medical professional.

In aspects, the present disclosure contemplates methods for preventing a brain tumor, e.g., without limitation, GBM, recurrence after surgical resection and chemotherapy and/or radiotherapy. In aspects, the present disclosure contemplates methods for preventing a brain tumor, e.g., without limitation, GBM, recurrence after treatment with an additional therapeutic agent and/or additional therapeutic modality, e.g., surgical resection, chemotherapy and/or radiotherapy using non-invasive brain stimulation, e.g., rTMS. In embodiments, the method of rTMS comprises repetitively applying a magnetic pulse to the scalp of a patient in need thereof, thereby affecting tumoral cells in the brain of the patient, wherein the magnetic pulse is applied (a) repetitively over the patient's brain; and (b) at a frequency of about 100 Hz to about 500,000 Hz and an intensity of about 5,000 milligauss to about 1,000,000 milligauss. In embodiments, methods of the present disclosure are suitable for home use and can be performed without the need for a medical professional.

In aspects, the present disclosure contemplates methods for slowing the progression of a brain tumor, e.g., without limitation, GBM. In aspects, the present disclosure contemplates methods for slowing the progression of a brain tumor, e.g., without limitation, GBM, using non-invasive brain stimulation, e.g., rTMS. In embodiments, the method of rTMS comprises repetitively applying a magnetic pulse to the scalp of a patient in need thereof, thereby affecting tumoral cells in the brain of the patient, wherein the magnetic pulse is applied (a) repetitively over the patient's brain; and (b) at a frequency of about 100 Hz to about 500,000 Hz and an intensity of about 5,000 milligauss to about 1,000,000 milligauss. In embodiments, methods of the present disclosure are suitable for home use and can be performed without the need for a medical professional.

In aspects, the present disclosure contemplates methods for treating recurrent a brain tumor, e.g., without limitation, GBM, recurrence using non-invasive brain stimulation, e.g., rTMS. In embodiments, the method of rTMS comprises repetitively applying a magnetic pulse to the scalp of a patient in need thereof, thereby affecting tumoral cells in the brain of the patient, wherein the magnetic pulse is applied (a) repetitively over the patient's brain; and (b) at a frequency of about 100 Hz to about 500,000 Hz and an intensity of about 5,000 milligauss to about 1,000,000 milligauss. In embodiments, methods of the present disclosure are suitable for home use and can be performed without the need for a medical professional.

In embodiments, the disclosure also provides a method to customize treatment by using finite element analysis simulation in conjunction with 3D data from a patient's MRI. In embodiments, the disclosure also provides a method employing a computational function in which the volume of the tumor is extracted from MRI data and then used as a template for finite element analysis of magnetic field with several coils. In embodiments, this computational function allows for alteration of parameters of intensity of the magnetic field and the set of coils from an array of coils to stimulate the specific location of the patient's tumor. In embodiments, methods include performing and/or utilizing information from one or more diagnostic procedures (e.g., MRI, CT scan, X-ray, etc.) to identify the location, or approximate location, of one or more tumors and/or metastasis for pinpointed rTMS targeting. In embodiments, the magnetic pulse is applied repetitively over the patient's skull as a function of the location of the one or more tumors and/or metastasis.

In embodiments, the present disclosure provides for rTMS treatment methods that treat or delay the progression of a brain tumor, e.g., without limitation, GBM. In embodiments, the treatment method improves and/or prevents brain tumor, e.g., without limitation, GBM, symptoms. In embodiments, the treatment method improves other symptoms related to a brain tumor, e.g., without limitation, GBM. Further embodiments of the present disclosure include methods for treating psychiatric symptoms linked to a brain tumor, e.g., without limitation, GBM, including, but not limited to, depression, anxiety, and cognitive decline.

In embodiments, rTMS methods herein generate a magnetic field within about 1 cm to about 2 cm from the skull, within about 1 cm to about 3 cm from the skull, within about 1 cm to about 4 cm from the skull, or within about 1 cm to about 5 cm from the skull. In embodiments, the positioning of the magnetic pulses is alterable, where the area and depth of the magnetic field is alterable as a function of the location of one or more malignant tissues.

In embodiments, rTMS methods herein reduce the migration and/or invasion phenotypes of cancerous cells and/or tissue, including the ability of cancerous cells and/or tissue to grow and spread. In embodiment, rTMS methods herein reduce the migration, invasion, and/or growth phenotypes of glioblastoma multiforme (GBM).

### Patient Selection

In embodiments, the present disclosure provides treatment of a patient that is afflicted with GBM as defined by the 2021 WHO classification criteria: Glioblastoma IDH-wildtype Grade IV or lower-grade IDH-wildtype astrocytoma with the presence of (one or in combination) (+7/−10), TERT mutations and/or EGFR amplification.

In embodiments, the present disclosure provides treatment of a patient that is afflicted with adult-diffuse gliomas including glioblastoma IDH-wildtype grade IV, and astrocytoma IDH-mutant grades II, III, IV. In embodiments, the present disclosure provides treatment.

The efficacy of treating GBM using methods and compositions of the present disclosure may be assessed by various methods. For example, endpoints based on tumor assessments, including disease free-survival (DFS), event-free survival (EFS), objective response rate (ORR), complete response (CR), time to progression (TTP), progression-free survival (PFS) and time to treatment failure (TTF).

### Additional Therapeutic Agents or Modalities and Combination Therapy

In embodiments the present treatment methods are used in combination with one or more of surgical resection, radiotherapy and alkylating chemotherapy. In embodiments administration of rTMS is combined with surgical resection of tumor. In embodiments administration of rTMS is combined with radiotherapy. In embodiments administration of rTMS is combined with an alkylating chemotherapy.

In embodiments administration of rTMS is combined with additional therapeutic agents and/or additional therapeutic modalities. Co-administration of the additional therapeutic agent and/or additional therapeutic modalities and the present rTMS may be simultaneous or sequential.

Further, the present methods provide for treatments of patients that are undergoing treatment with one or more additional therapeutic agents and/or additional therapeutic modalities. In embodiments, the additional therapeutic agent and/or additional therapeutic modality is as described herein.

In one embodiment, the additional therapeutic agent and/or additional therapeutic modality and the rTMS are administered to a patient simultaneously. The term “simultaneously” as used herein, means that the additional therapeutic agent and/or additional therapeutic modality and the rTMS are administered with a time separation of no more than about 60 minutes, such as no more than about 30 minutes, no more than about 20 minutes, no more than about 10 minutes, no more than about 5 minutes, or no more than about 1 minute.

In a further embodiment, the additional therapeutic agent and the rTMS are administered to a patient simultaneously but the release of the additional therapeutic agent from its respective dosage form and the rTMS may occur sequentially.

Co-administration does not require the additional therapeutic agent and the rTMS to be administered simultaneously, if the timing of their administration is such that the pharmacological activities of the additional therapeutic agent and the administration of TMS overlap in time. For example, the additional therapeutic agent and the rTMS can be administered sequentially. The term “sequentially” as used herein means that the additional therapeutic agent and the rTMS are administered with a time separation of more than about 60 minutes. For example, the time between the sequential administration of the additional therapeutic agent and the administration of rTMS can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent being administered. Either the additional therapeutic agent or the rTMS may be administered first.

In embodiments, the present disclosure contemplates rTMS methods used in tandem with one or more additional therapeutic agents and/or additional therapeutic modalities. In embodiments, the rTMS methods of the present disclosure obviate the need for treatment with one or more additional therapeutic agents. In embodiments, the additional therapeutic agent is selected from chemotherapeutic agents, immunotherapy or radiotherapy. In embodiments, the rTMS methods of the present disclosure allow for reduction of the dosage or frequency of administration of one or more therapeutic agents and/or additional therapeutic modalities. In embodiments, the additional therapeutic agent is a chemotherapy.

In embodiments, the additional therapeutic agent is or comprises a treatment for a brain tumor, e.g., without limitation, GBM, including, but not limited to, temozolomide. In embodiments, the rTMS treatment of the present disclosure is substantially free of adverse effects, optionally selected from epileptic seizures, nausea, headache, vagal response, musculoskeletal pain, scalp petechial rash, scalp pain, edema, or bruising.

In embodiments, the additional therapeutic agent inhibits the vascular endothelial growth factor, including, but not limited to, bevacizumab.

In embodiments, the additional therapeutic agent is or comprises an alkylating chemotherapy. In embodiments, the alkylating chemotherapy is temozolomide.

In embodiments, the additional therapeutic agent is or comprises an immunotherapy specifically designed to target a brain tumor, e.g., without limitation, GBM.

In embodiments, the additional therapeutic agent is or comprises a radiotherapy.

In embodiments, the present disclosure contemplates rTMS methods used in tandem with one or more additional therapeutic agents and/or additional therapeutic modality is or comprises cytoreductive surgery, laser interstitial thermal therapy (LITT), the use of fluorescence detection techniques and intraoperative MRI (iMRI) to maximize resection, MRI-guided Radiation Therapy, microtubule-targeting agents (MTAs), and focused ultrasound (FUS) for BBB disruption to facilitate targeted drug delivery.

### Definitions

As used herein, “a,” “an,” or ““they”” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses, is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of the ordinary skills in the art, in embodiments, activity is decreased, and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of a stimulus, relative to the absence of such stimulus.

As used herein, the word “include,” and its variants, is intended to be non-limiting, such that recitation of items in a list is not to the exclusion of other like items that may also be useful in the treatments and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

An effective amount of the treatment as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

In embodiments, the effect will result in a quantifiable change of at least about 10%, at least about 20%, at least about 30%, at least about 50%, at least about 70%, or at least about 90%. In embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: High Frequency and Low Intensity Transcranial Magnetic Stimulation for the Treatment of GBM: An In Vitro Study

The objective of this study is to evaluate the effects of a 5-day intervention with high frequency and low intensity TMS (HFLI TMS) on cell migration, cell proliferation, and cell death of GBM cells in vitro. The study will be performed using two different cell lines: GL261, a mouse glioblastoma cell line and U87 MG, a human glioblastoma cell line. Cell migration and cell proliferation will be used as measurements to determine efficacy of intervention on the GBM cell lines. These parameters will be evaluated using a simple technique known as “scratch-wound assay”; immunofluorescence will also be used to detect expression of cell proliferation (Ki67), cytoskeletal changes (actin) and cell death (live/dead cell assay).

To evaluate cell migration, GL261 and U87 cultured cells will be plated at a specific cell density onto chambered cell culture slides and a scratch-wound will be done on the cell monolayer, using a 200 μL sterile pipette tip. Cells will be exposed to 200 or 2000 Hz HFLI TMS for 30 minutes over 5 consecutive days and migration of cells into the cell-free scratch-wound will be assessed as a measure of invasiveness. Cells will be kept at room temperature for the duration of the stimulation and returned to the incubator (37° C., 5% CO2) once the session is over.

To evaluate cell proliferation, cell death and changes in the cytoskeleton, GL261 and U87 cultured cells will be plated at a specific cell density onto chambered cell culture slides and will be exposed to HFLI TMS for 30 minutes over 5 consecutive days. Using immunofluorescence, the expression of Ki67 and actin will be determined as a measure of cell proliferation and changes in cell morphology, respectively. To assess cell death, a fluorescent live/dead cell assay will be used to detect the percentage of tumor cell death after the intervention.

### Example 2: High Frequency and Low Intensity Transcranial Magnetic Stimulation for the Treatment of GBM: An In Vivo Study

An objective of this study is to evaluate the effects of a 21-day intervention with high frequency and low intensity TMS (HFLI TMS) on the growth and microenvironment of GBM, and on overall survival of the animal model; the safety of the intervention will also be assessed. The study is performed on an orthotopic xenograft mouse model of human GBM. Overall survival of mice is used as an objective measurement to determine efficacy of intervention and safety; histological assessment of the mouse brain is also used to establish the impact of the intervention on tumor growth (cell proliferation and cell death), on the tumor microenvironment (neuroinflammation) and also provides evidence of the safety of the intervention.

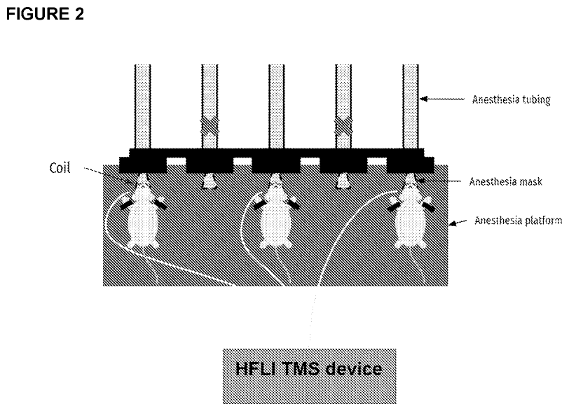

The athymic nude mouse is used to generate the orthotopic xenograft model by injecting U87 GBM human cells into the mouse striatum, using a stereotaxic apparatus. This allows the development of a tumor of GBM characteristics over the course of seven days. On the 8th day, injected mice are divided into three groups, one group receives HFLI TMS at a frequency of 200 Hz, while a second group of mice is exposed to 2000 Hz, and a third group receives sham stimulation (FIG. 2). The intervention is composed of 45 min daily sessions of HFLI TMS for 21 consecutive days. Mice are anesthetized during the HFLI TMS sessions and monitored throughout the study. If the animal's clinical condition reveals clear signs of suffering such as a 10% weight loss over 24 hours or prostration, analgesia is provided for 72 hours, renewed every 8 to 12 hours. If no improvement is observed, the protocol is stopped prematurely.

Mice re euthanized on day 21. The brains are processed for histological assessment. Results re reported as overall survival after 21 days; histological changes are evaluated using immunohistochemistry on frozen or formalin-fixed mouse brains to detect expression of proliferation (Ki67), neuroinflammation (GFAP, CD68) and cell death (BCL2, caspase-3) markers after the HFLI TMS 21-day intervention. The histological data provides information, e.g., on the efficacy of the intervention in preventing GBM cells proliferation, neuroinflammation and in promoting GBM cell death.

### Example 3: Investigating the Effect of rTMS on Blood-Brain Barrier (BBB) Permeability In Vivo

An in vivo mouse model was used to evaluate the use of rTMS to open the BBB. Ten wild-type C57BL/6 mice where anesthetized and received an intravenous (IV) injection of a 2% Evans Blue dye (in a vehicle of 0.9% NaCl). Evans Blue dye (also referred to as EBD or T-1824) is a non-toxic, azo-based injectable dye that presents visually with a dark blue/brown color. The dye has an affinity for serum albumin and is generally used to assess the permeability of the BBB in vivo. Most tissues will present darker, but neural tissue generally remains unstained. Mice injected with Evans Blue dye were then administered rTMS or a sham treatment and a histological analysis of the brain was performed to evaluate changes in permeability of the BBB. rTMS was administered according to Table 1.

TABLE 1
Study design for in vivo administration of rTMS. All
mice were female, wild-type (C56BL/7) genotype.

| Mouse No. | Injection | Animal Position |
|---|---|---|
| 1 | Evans Blue + 1 hr. rTMS | Sphinx |
| 2 | Evans Blue + 1 hr. sham | Sphinx |
| 3 | Evans Blue + 1 hr. rTMS | Sphinx |
| 4 | Evans Blue + 1 hr. sham | Sphinx |
| 5 | Evans Blue + 1 hr. rTMS | Left Lateral |
| 6 | Evans Blue + 1 hr. sham | Left Lateral |
| 7 | Evans Blue + 1 hr. rTMS | Right Lateral |
| 8 | Evans Blue + 1 hr. sham | Right Lateral |
| 9 | Evans Blue + 30 min rTMS | Left Lateral |
| 10 | Evans Blue + 30 min sham | Left Lateral |

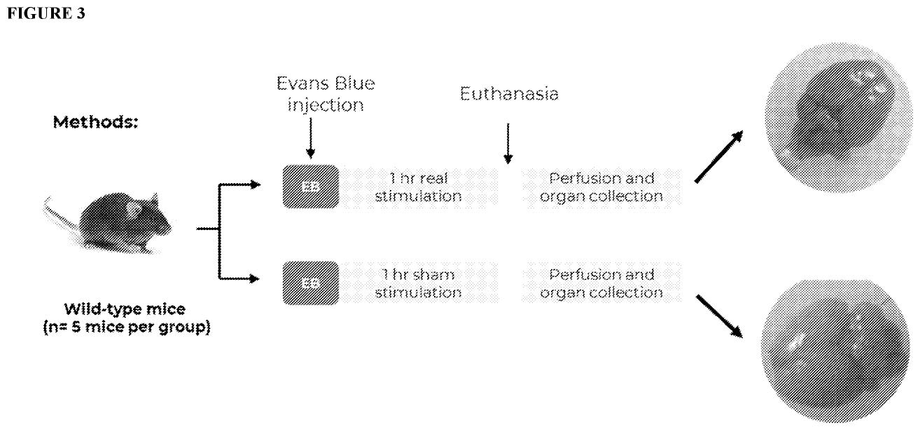

The dye was administered as 100 μL 2% Evans blue (in 0.9% NaCl) via retro orbital injection. Mice were then administered rTMS or a sham (no magnetic field), e.g., as shown in the method overview depicted in FIGS. 2 and 3 for either 1 hr. or 30 min. Mice were euthanized, perfused with 10 mL PBS to expel blood from the organs, and analyzed for the presence of hemorrhage and lesion, e.g., as shown in the representative imaging in FIG. 3. Mice presented as lacking hemorrhage, lesion, or any signs of tissue damage.

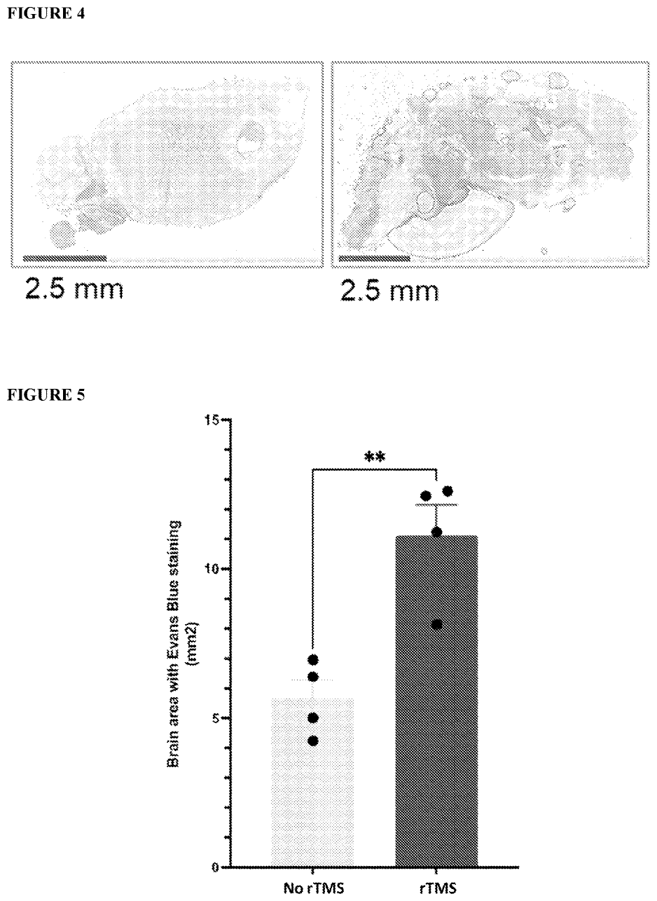

Brains were then submerged in 20% sucrose for 2 hr. and embedded in optimal cutting temperature compound (OCT compound) and frozen. The brain was then sectioned at 16 μm thickness, rinsed with PBS, and mounted for scanning at 40× magnification. Section imaging was performed using a digital slide scanner. An illustrative comparison demonstrating the efficacy of uptake of the Evans Blue dye through the BBB from rTMS is shown in FIG. 4, with Evans blue staining appearing as the darker regions within the tissue, as well as the outlining of the brain microvasculature.

An imaging software was used to evaluate the pixelate density and area of staining in brain sections for calculating a comparison of Evans Blue dye uptake into the brain tissue. A statistical analysis was performed, e.g., as summarized in Table 2. As enumerated in FIG. 5, the data demonstrated a statistically significant difference between the sham and rTMS groups, with a mean stained area of 5.665 mm² and 11.12 mm², respectively, with a p-value of 0.004, indicating a statistically significant increase in BBB permeability and opening from the rTMS use.

TABLE 2
Statistical analysis of BBB opening between rTMS and sham
treatment. Sample size is 4 per sham and rTMS groups.

| Statistical Test | Metric | Value |
|---|---|---|
| Unpaired | P value | 0.004 |
| t-test | Significantly Different (<0.05) | Yes |
| | One-tailed or two-tailed P value | Two-tailed |
| | T, df | T = 4.516; df = 6 |
| Comparison of | Mean sham | 5.665 mm² |
| Difference | Mean rTMS | 11.12 mm² |
| | Difference between means ± SEM | 5.456 ± 1.209 mm² |
| | 95% Confidence Interval | 2.498-8.413 |
| | R² | 0.7725 |
| F test to | F, DFn, Dfd | 2.775, 3, 3 |
| compare variances | P value | 0.4242 |
| | P value summary | ns |
| | Significantly difference (P < 0.05) | no |

The data demonstrated, inter alia, that rTMS treatment greatly increased the permeability of the BBB, resulting in a statistically significant (nearly 2×) increase, and that this increase in BBB permeability is suitable for trafficking molecules to cross the BBB, e.g., such as the dye molecule and other small molecules.

### Example 4: Investigating the Effect of rTMS on Blood-Brain Barrier (BBB) Permeability and Glial Integrity In Vivo

An in vivo mouse model was used to evaluate the effect of rTMS on glial cells in the BBB and to visualize in vivo BBB permeability of blood vessels. CXCR1 mice with endogenous green fluorescent protein (GFP)-expressing microglial cells where anesthetized and received a systemic injection of rhodamine dextran (e.g., producing red fluorescence). Rhodamine dextran is commonly used to image the cerebral vasculature to observe changes in blood flow dynamics and blood-brain barrier dynamics in vivo.

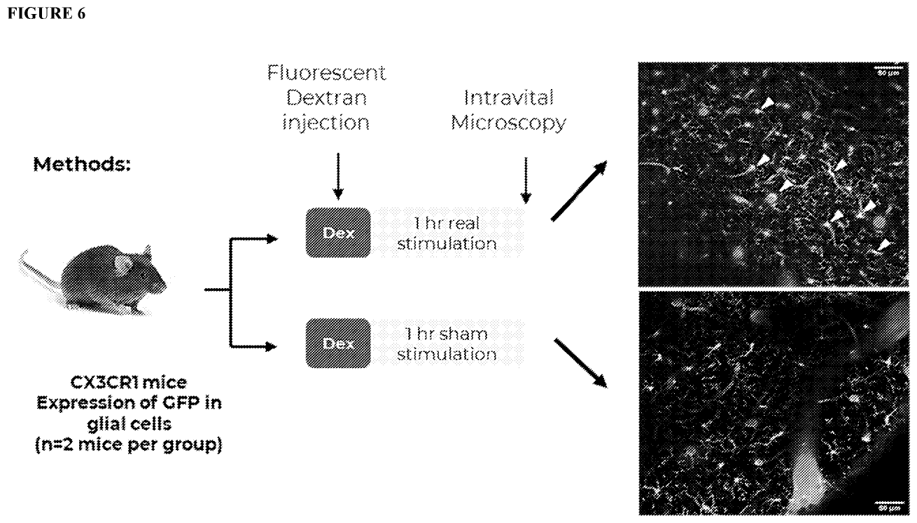

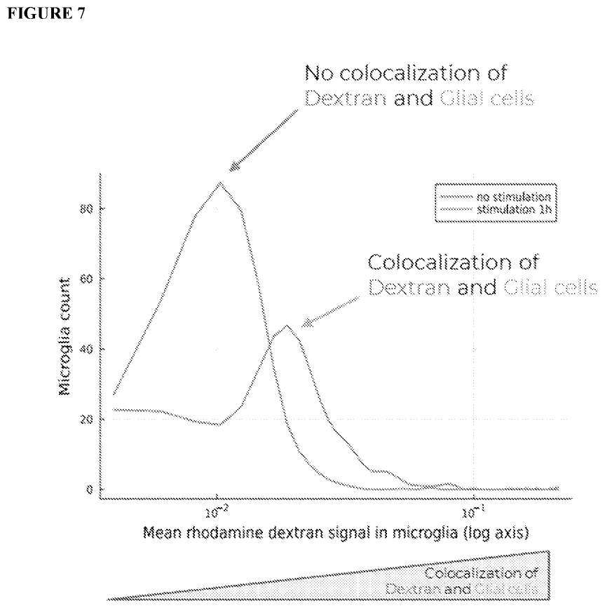

Mice were then administered rTMS or a sham treatment (no magnetic field) for 1 hr., e.g., as shown in the method overview depicted in FIG. 6. Representative in vivo fluorescence confocal microscopy imaging (e.g., as shown in FIG. 6) demonstrated a robust increase in the colocalization of rhodamine fluorescence with the GFP fluorescence of glial cells after rTMS exposure, indicating increased BBB vasculature permeability (e.g., leaky vasculature). FIG. 7 summarizes the quantitative results of imaging, which demonstrated that rTMS increased rhodamine permeation into microglial cells as demonstrated by the increased colocalization of fluorescence, concomitant with increased permeability (e.g., the formation of microlesions) in the vasculature of the BBB. These effects were observed to be transient and reversible, causing no overall deleterious effects in the mice.

These data illustrate, inter alia, that rTMS functioned via a mechanism of increasing BBB permeability by inducing reversible leakage in the microvasculature, and allowed molecules to traffic across the microvasculature of the BBB in vivo.

### Example 5: Investigating the Effect of rTMS on Glioblastoma (GBM) In Vitro

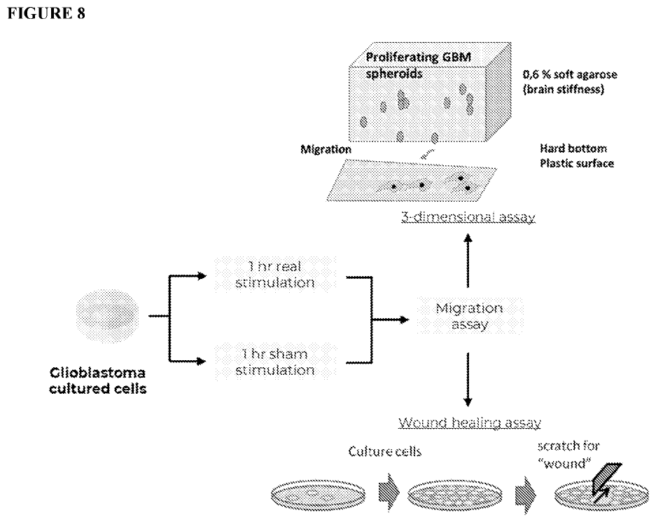

A glioblastoma multiforme (GBM) cell line was used to assess the effects of rTMS on migration and invasion phenotypes. GBM cells were cultured and exposed to either rTMS or a sham (no magnetic field) for 1 hr., e.g., as shown in the method overview depicted in FIG. 8. Treated cells were then seeded for evaluation using two distinct migration and invasion assays, e.g., 2-dimensional and 3-dimensional growth modalities to more closely replicate in vivo growth conditions.

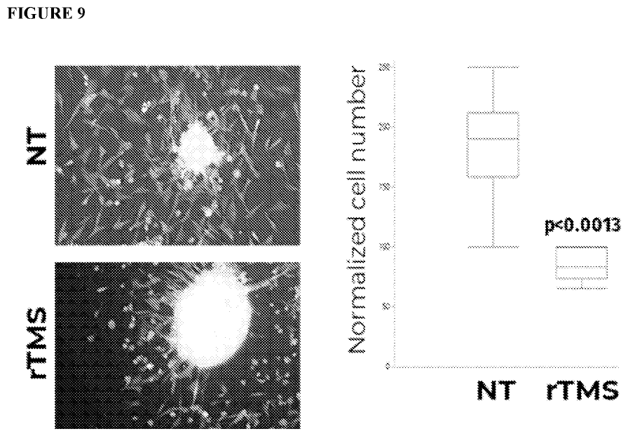

The results of the 3-dimensional growth assay are shown in FIG. 9. Cells were seeded into a 0.6% agarose matrix which is consistent with the stiffness of brain tissue. FIG. 9 shows representative fluorescence confocal microscopy imaging and a graphical representation quantifying the results of rTMS on the effect of glioblastoma cells to form spheroids using a 3-dimensional spheroid growth assay. Spheroids were compared for their number of total cells. rTMS led to a statistically significant decrease in the size of 3D spheroid formation compared to sham treatment (p<0.013).

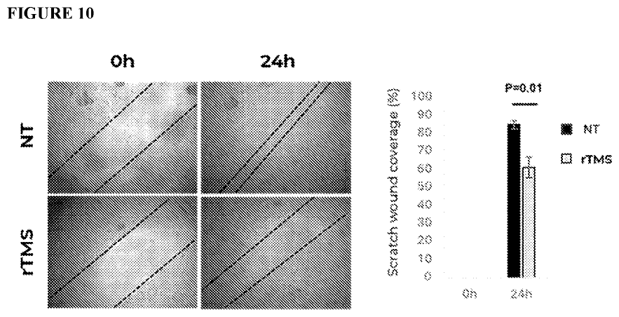

The results of the 2-dimensional wound healing assay are shown in FIG. 10. Cells were grown to confluency in a cell monolayer where a scratch “wound” was made in the cell monolayer. The ability of the cells to close the wound over a 24 hour time point was evaluated as a quantitative measure of invasion and migration. FIG. 10 shows representative confocal microscopy imaging and a graphical representation quantifying the effect of rTMS on the wound healing. rTMS was shown to lead to a statistically significant decrease in invasion and wound healing of glioblastoma cells at 24 hrs. compared to sham treatment (p=0.01).

These data illustrate, inter alia, that rTMS decreased the migration, invasion, and growth of glioblastoma cells both under cell monolayer conditions and 3-dimensional growth conditions which recapitulate in vivo brain cancer growth conditions. Moreover, the data suggest that rTMS induced leakage in the BBB microvasculature to disrupt cancer cell phenotypes without inducing deleterious effects on healthy tissue.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### REFERENCES

Alzial G, Renoult O, Paris F, Gratas C, Clavreul A & Pecqueur C (2022) Wild-type isocitrate dehydrogenase under the spotlight in glioblastoma. Oncogene 41, 613-621.
Chen X, Wanggou S, Bodalia A, Zhu M, Dong W, Fan J J, Yin W C, Min H-K, Hu M, Draghici D, Dou W, Li F, Coutinho F J, Whetstone H, Kushida M M, Dirks P B, Song Y, Hui C-C, Sun Y, Wang L-Y, Li X & Huang X (2018) A Feedforward Mechanism Mediated by Mechanosensitive Ion Channel PIEZO1 and Tissue Mechanics Promotes Glioma Aggression. Neuron 100, 799-815.e7.
Grochans S, Cybulska A M, Simińska D, Korbecki J, Kojder K, Chlubek D & Baranowska-Bosiacka | (2022) Epidemiology of Glioblastoma Multiforme-Literature Review. Cancers 14, 2412.
Ishihara S & Haga H (2022) Matrix Stiffness Contributes to Cancer Progression by Regulating Transcription Factors. Cancers 14, 1049.
Le Rhun E, Preusser M, Roth P, Reardon D A, van den Bent M, Wen P, Reifenberger G & Weller M (2019) Molecular targeted therapy of glioblastoma. Cancer Treat. Rev. 80, 101896.
Leao D J, Craig P G, Godoy L F, Leite C C & Policeni B (2020) Response Assessment in Neuro-Oncology Criteria for Gliomas: Practical Approach Using Conventional and Advanced Techniques. AJNR Am. J. Neuroradiol. 41, 10-20.
Li C, Rezania S, Kammerer S, Sokolowski A, Devaney T, Gorischek A, Jahn S, Hackl H, Groschner K, Windpassinger C, Malle E, Bauernhofer T & Schreibmayer W (2015) Piezo1 forms mechanosensitive ion channels in the human MCF-7 breast cancer cell line. Sci. Rep. 5, 8364.
Louis D N, Ellison D W, Brat D J, Aldape K, Capper D, Hawkins C, Paulus W, Perry A, Reifenberger G, Figarella-Branger D, von Deimling A & Wesseling P (2019) cIMPACT-NOW: a practical summary of diagnostic points from Round 1 updates. Brain Pathol. 29, 469-472.
Louis D N, Perry A, Wesseling P, Brat D J, Cree I A, Figarella-Branger D, Hawkins C, Ng H K, Pfister S M, Reifenberger G, Soffietti R, von Deimling A & Ellison D W (2021) The 2021 WHO Classification of Tumors of the Central Nervous System: a summary. Neuro-Oncol. 23, 1231-1251.
Melhem J M, Detsky J, Lim-Fat M J & Perry J R (2022) Updates in IDH-Wildtype Glioblastoma. Neurotherapeutics 19, 1705-1723.
Miller K D, Ostrom Q T, Kruchko C, Patil N, Tihan T, Cioffi G, Fuchs H E, Waite K A, Jemal A, Siegel R L & Barnholtz-Sloan J S (2021) Brain and other central nervous system tumor statistics, 2021. CA. Cancer J. Clin. 71, 381-406.
Motomura K, Takeuchi H, Nojima I, Aoki K, Chalise L, lijima K, Wakabayashi T & Natsume A (2020) Navigated repetitive transcranial magnetic stimulation as preoperative assessment in patients with brain tumors. Sci. Rep. 10, 9044.
Rominiyi O, Vanderlinden A, Clenton S J, Bridgewater C, Al-Tamimi Y & Collis S J (2021) Tumour treating fields therapy for glioblastoma: current advances and future directions. Br. J. Cancer, 124, 697-709.
Rong L, Li N & Zhang Z (2022) Emerging therapies for glioblastoma: current state and future directions. J. Exp. Clin. Cancer Res. 41, 142.
van Solinge T S, Nieland L, Chiocca E A & Broekman M L D (2022) Advances in local therapy for glioblastoma-taking the fight to the tumour. Nat. Rev. Neurol. 18, 221-236.
Sprugnoli G, Monti L, Lippa L, Neri F, Mencarelli L, Ruffini G, Salvador R, Oliveri G, Batani B, Momi D, Cerase A, Pascual-Leone A, Rossi A, Rossi S & Santarnecchi E (2019) Reduction of intratumoral brain perfusion by noninvasive transcranial electrical stimulation. Sci. Adv. 5, eaau9309.
Torp S H, Solheim O & Skjulsvik A J (2022) The WHO 2021 Classification of Central Nervous System tumours: a practical update on what neurosurgeons need to know—a minireview. Acta Neurochir. (Wien) 164, 2453-2464.
U.S. Department of Health and Human Services, Food and Drug Administration (2018) Clinical Trial Endpoints for the Approval of Cancer Drugs and Biologics.
Weller M, van den Bent M, Preusser M, Le Rhun E, Tonn J C, Minniti G, Bendszus M, Balana C, Chinot O, Dirven L, French P, Hegi M E, Jakola A S, Platten M, Roth P, Rudà R, Short S, Smits M, Taphoorn M J B, von Deimling A, Westphal M, Soffietti R, Reifenberger G & Wick W (2021) EANO guidelines on the diagnosis and treatment of diffuse gliomas of adulthood. Nat. Rev. Clin. Oncol. 18, 170-186.
Wullkopf L, West A-K V, Leijnse N, Cox T R, Madsen C D, Oddershede L B & Erler J T (2018) Cancer cells' ability to mechanically adjust to extracellular matrix stiffness correlates with their invasive potential. Mol. Biol. Cell, 29, 2378-2385.
Zhou W, Liu X, van Wijnbergen J W M, Yuan L, Liu Y, Zhang C & Jia W (2020) Identification of PIEZO1 as a potential prognostic marker in gliomas. Sci. Rep. 10, 16121.

What is claimed is:

1. A repetitive transcranial magnetic stimulation (rTMS) method for treating or preventing a brain cancer comprising repetitively applying a magnetic pulse with one or more coils configured to emit a magnetic field to the scalp of a patient in need thereof thereby affecting cancer cells in the brain of the patient, wherein the magnetic pulse is applied:
repetitively over the patient's brain; and
at a frequency of about 10 Hz to about 500,000 Hz and an intensity of about 0.0005 Tesla to about 0.1 Tesla.

2. A repetitive transcranial magnetic stimulation (rTMS) method for slowing or preventing the recurrence of brain cancer comprising repetitively applying a magnetic pulse with one or more coils configured to emit a magnetic field to the scalp of a patient in need thereof thereby affecting cancer cells in the brain of the patient, wherein the magnetic pulse is applied:
repetitively over the patient's brain; and
at a frequency of about 10 Hz to about 500,000 Hz and an intensity of about 0.0005 Tesla to about 0.1 Tesla.

3. The method of either claim 1 or 2, wherein the magnetic pulse is applied at a frequency of about 100 to about 600 Hz.

4. The method of claim 3, wherein the magnetic pulse is applied at a frequency of about 200 Hz.

5. The method of any one of the previous claims, wherein the magnetic pulse is applied at an intensity of about 0.001 Tesla to 0.1 Tesla.

6. The method of any one of the previous claims, wherein the magnetic pulse applied at an intensity of about 0.01 Tesla.

7. The method of any one of the previous claims, wherein the brain cancer is a brain tumor and/or metastasis.

8. The method of claim 7, wherein the brain tumor is an astrocytoma.

9. The method of claim 7, wherein the brain tumor is a glioblastoma.

10. The method of claim 9, wherein the glioblastoma is glioblastoma multiforme (GBM).

11. The method of any one of claim 9 or 10, wherein the glioblastoma is a primary glioblastoma or a secondary glioblastoma.

12. The method of any one of claim 7-11, wherein the brain tumor localizes to and/or originates in the supratentorial compartment, frontal lobe, temporal lobe, parietal lobe, occipital lobes, brainstem and/or cerebellum.

13. The method of any one of the previous claims, wherein the method further comprises surgical resection, radiotherapy, and/or administration of chemotherapy.

14. The method of any one of the previous claims, wherein the patient has undergone or is undergoing surgical resection, radiotherapy, and/or administration chemotherapy.

15. The method of claim 13 or 14, wherein the chemotherapy is alkylating chemotherapy.

16. The method of any one of the previous claims, wherein the rTMS treatment is self-applied.

17. The method of any one of the previous claims, wherein the magnetic pulse is applied using a device, wherein the device is suitable for conducting electric current through the one or more coils to emit the magnetic field.

18. The method of claim 17, wherein the device is suitable for home use.

19. The method of claim 17, wherein the device is portable.

20. The method of any one of claims 17-19, wherein the device is wearable.

21. The method of any one of the previous claims, where in the patient is afflicted with glioblastoma multiforme (GBM).

22. The method of any one of the previous claims, wherein the patient is afflicted with one or more symptoms selected from intracranial hypertension, headaches, seizures, sensory disturbances, neurocognitive symptoms, and motor symptoms.

23. The method of any one of the preceding claims, wherein the patient presents as having the diagnosis of GBM according to The 2021 World Health Organization (WHO) Classification of Tumors of the Central Nervous System.

24. The method of any one of the preceding claims, wherein the method stimulates tumor cells within about 1 cm to about 2 cm from the skull, within about 1 cm to about 3 cm from the skull, within about 1 cm to about 4 cm from the skull, or within about 1 cm to about 5 cm from the skull.

25. The method of any one of the preceding claims, wherein the magnetic pulse is applied repetitively over the patient's skull as a function of a location of a tumor.

26. The method of any one of the preceding claims, further comprising identifying a location of a tumor prior to administering the rTMS and/or after applying the rTMS, optionally using one or more of magnetic resonance imaging (MRI), computed tomography (CT) scan, and X-ray.

27. The method of any one of the preceding claims, wherein the method reduces, impairs, and/or decreases tumor growth, migration, invasiveness, and/or spread throughout the patient's brain.

28. The method of any one of the preceding claims, wherein the method prevents or delays the recurrence of GBM.

29. The method of any one of the preceding claims, wherein the treatment improves symptoms of the patient, optionally symptoms of GBM.

30. The method of any one of the preceding claims, wherein the treatment curtails or prevents exacerbation of GBM.

31. The method of any one of the preceding claims, wherein the treatment is used in tandem with one or more additional therapeutic agents.

32. The method of any one of the preceding claims, wherein the rTMS treatment obviates the need for treatment with one or more additional therapeutic agents and/or additional therapeutic modalities; and/or wherein the rTMS treatment allows for reduction of the dosage or frequency of administration of one or more additional therapeutic agents and/or additional therapeutic modalities.

33. The method of any one of the preceding claims, wherein the rTMS treatment is substantially free of adverse effects, optionally selected from epileptic seizures, nausea, headache, vagal response, musculoskeletal pain, scalp petechial rash, scalp pain, edema, or bruising.
